(19) United States
(12) Patent Application Publication
(10) Pub. No.: US 2021/0244033 A1
(43) Pub. Date: Aug. 12, 2021

(54) METHODS AND COMPOSITION FOR CONTROLLING MAP INFECTION AND CONTAMINATION

(71) Applicant: MAP/PATH LLC, St. Paul, MN (US)

(72) Inventor: John E. Haaland, Saint Paul, MN (US)

(73) Assignee: MAP/PATH LLC, St. Paul, MN (US)

(21) Appl. No.: 17/204,432

(22) Filed: Mar. 17, 2021

Related U.S. Application Data
(63) Continuation of application No. PCT/US2019/051448, filed on Sep. 17, 2019.
(60) Provisional application No. 62/733,019, filed on Sep. 18, 2018.

(51) Int. Cl. A01N 63/40; A01N 63/20; A61K 9/00; A61K 35/76; A61K 35/74; A61P 31/04

(52) U.S. Cl. CPC A01N 63/40; A01N 63/20; A61P 31/04; A61K 35/76; A61K 35/74; A61K 9/0056

(57) ABSTRACT

Provided herein, in some embodiments, are methods and composition for controlling Mycobacterium avium subspecies paratuberculosis (MAP) infection and contamination.

### RELATED APPLICATIONS

This application is a continuation of International Application No. PCT/US2019/051448, filed Sep. 17, 2019, which claims the benefit under 35 U.S.C. § 119(e) of U.S. provisional application No. 62/733,019, filed Sep. 18, 2018, each of which is incorporated by reference herein in its entirety.

### BACKGROUND

Mycobacterium avium subspecies paratuberculosis (MAP) causes Johne's disease in cattle and other ruminants. It is also suspected as a causative agent in Crohn's disease in humans. MAP can survive pasteurization, which has raised human health concerns due to the widespread nature of MAP in modern dairy herds. The mycobacterium is heat resistant and is capable of sequestering itself inside macrophages (white blood cells), which may contribute to its persistence in dairy products, such as milk. It has also been reported to survive chlorination in municipal water supplies. MAP is not susceptible to anti-tuberculosis drugs (which can generally kill Mycobacterium tuberculosis). While MAP is susceptible to antibiotics such as rifabutin and clarithromycin, used to treat Mycobacterium avium disease, the capacity of these antibiotics to eradicate MAP infection in vivo has not been established.

### SUMMARY

Provided herein, in some aspects, are methods of controlling dissemination (e.g., infection and/or contamination) of Mycobacterium avium subspecies paratuberculosis (MAP) using a “cocktail” that can potentially eradicate MAP in both morphological states—capsulated/dormant and uncapsulated/active. In some aspects of the present disclosure, the methods comprise applying to food, water, ground, and/or excrement (e.g., present in fertilizer) a composition comprising a mycobacteriophage (e.g., a Cluster K mycobacteriophage, such as mycobacteriophage TM4, see, e.g., Pope W H et al. PLOS One 2011; 6(10):e26750, 1-22) and/or an avirulent mycobacterium (e.g., Mycobacterium smegmatis) transfected with the mycobacteriophage in an amount effective for lysing capsulated (e.g., extracellular) MAP and uncapsulated (e.g., intracellular) MAP. In some embodiments, the food, water, ground, and/or excrement is contaminated with MAP or is at risk of contamination with MAP.

Other aspects of the present disclosure provide methods and compositions for controlling MAP infection, e.g., in ruminants and humans. In some embodiments, the methods comprise delivering to a subject infected with MAP or at risk of infection with MAP a composition comprising a mycobacteriophage (e.g., Cluster K mycobacteriophage, such as mycobacteriophage TM4), avirulent mycobacterium (e.g., Mycobacterium smegmatis) transfected with the mycobacteriophage, or a combination thereof in an amount effective for lysing capsulated (e.g., extracellular) MAP and uncapsulated (e.g., intracellular) MAP.

Thus, provided herein are methods that comprise contacting a sample, contaminated with MAP, with a composition comprising a mycobacteriophage (e.g., Cluster K mycobacteriophage, such as mycobacteriophage TM4), an avirulent mycobacterium (e.g., Mycobacterium smegmatis) transfected with the mycobacteriophage, or a combination thereof in an amount effective for lysing capsulated (e.g., extracellular) MAP and uncapsulated (e.g., intracellular) MAP; and lysing capsulated MAP and uncapsulated MAP in the sample.

### BRIEF DESCRIPTION OF THE DRAWINGS

FIG. 1 depicts a growth curve showing the quantification of mycobacteriophage TM4 by qPCR and a reduction in Mycobacterium smegmatis growth.

FIG. 2 depicts graphs showing active growth of three concentration of Mycobacterium avium subspecies paratuberculosis (MAP)-spiked control cultures in select media in TREK bottles.

### DETAILED DESCRIPTION

For over 100 years an incurable infectious disease has been killing cattle, goats, sheep, camels, buffalo, deer, and elk. Intensive research has been done over the past 35 years to cure this disease, but without success. The responsible bacterium is Mycobacterium avium subspecies paratuberculosis (MAP). MAP is related to the bacteria that cause tuberculosis and leprosy. There is a long latent period during which the infected animal sheds the bacterium (in milk and excrement/feces) before it becomes clinically ill. The disease is then managed by farmers and veterinarians by culling (killing) infected members of the herd. For the dairy industry the cost of Johne's Disease (JD) is about $270 million dollars per year in North America. These animals are part of our food supply (e.g. dairy products and hamburger) and the MAP pathogen, responsible for JD, is found in them. Being heat resistant, it survives pasteurization of milk, baby's food and dehydrated baby and calf food formulas. In beef cattle, as many as 39% of harvested cattle may be infected by it, resulting in it being present in our steaks and hamburger. This same pathogen also is implicated in many human diseases. The majority of global researchers in this area say that MAP will be soon be recognized as a zoonotic disease. If no fully effective risk mitigation is available, this could result in considerable demand decrease and a potential reduction in product supply as a result of regulatory measures and public demand that MAP not be in the food chain and be labeled accordingly. Provided herein are methods and compositions for controlling MAP infection and contamination.

Mycobacterium avium Subspecies Paratuberculosis (MAP)

Mycobacterium avium subspecies paratuberculosis (MAP) is an obligate pathogenic bacterium in the genus Mycobacterium. It is the etiological agent of Johne's disease (JD) (paratuberculosis), a chronic granulomatous enteritis of ruminants, and is suspected as a causative agent in human Crohn's disease and rheumatoid arthritis. The genome of MAP strain K-10 was sequenced in 2005 and found to include a single circular chromosome of 4,829,781 base pairs, and to encode 4,350 predicted open reading frames (ORFs), 45 tRNAs, and one rRNA operon (Li L et al. PNAS USA 2005; 102(35):12344). The MAP type strain is ATCC 19698 (equivalent to OP 103963 or DSM 44133) (Thorel M et al. Int J Syst Bacteriol 1990; 40(3):254-60).

Because of its inability to produce mycobactin (a chemical needed to transport iron), MAP can grow only inside eukaryote (e.g., animal cells) where it “steals” iron from its host's cells, most often immune cells called macrophages. This means infected animals, humans and eukaryote microbes are the only place in nature where its active (e.g., uncapsulated) growth and multiplication of MAP can occur.

Their long-term survival depends on their unique spore-like capsulated morphology. Mycobacterium avium subspecies paratuberculosis (MAP) can exist in (and switch between) two forms: an ‘extracellular’ form (i.e., adapted to live outside of other cells) with an outer ‘capsule’ and an ‘intracellular’ form (i.e., adapted to live inside other eukaryote cells) that sheds its capsule and adapts its actively replicating form after being taken up into white blood cells in the animal's bloodstream and tissues.

Active replicating MAP transforms into a dormant capsulated endospore under stress (e.g., starvation, temperature, pH, chemical environment) that is almost indestructible. Under favorable conditions in a host it transforms its morphology to allow replicating again. See e.g., Identification and Characterization of a Spore-Like Morphotype in Chronically Starved Mycobacterium avium Subsp. Paratuberculosis Cultures; Elise A. Lamont, John P. Bannantine, Aníbal Armién, Don Sanjiv Ariyakumar, Srinand Sreevatsan; Published: Jan. 24, 2012.

The present disclosure provides, in some aspects, methods and compositions for controlling MAP infection and contamination. In some embodiments, the methods comprise delivering to a subject infected with MAP, or at risk of infection with MAP, a composition comprising a Cluster K mycobacteriophage, such as TM4 (see, e.g., Pope W H et al. PLOS One 2011; 6(10):e26750) and/or an avirulent mycobacterium transfected with a Cluster K mycobacteriophage. In some embodiments, a composition comprises a mixture of mycobacteriophage and avirulent bacteria transfected with mycobacteriophage. In some embodiments, a composition comprises a mixture of Cluster K mycobacteriophage and avirulent bacteria transfected with Cluster K mycobacteriophage. In some embodiments, a composition comprises a mixture of mycobacteriophage TM4 and Mycobacterium smegmatis transfected with mycobacteriophage TM4. Without being bound by theory, it is thought that the “free” mycobacteriophage of the compositions infect and lyse capsulated (e.g., extracellular, inactive and dormant, in the form of an endospore) MAP, while the avirulent bacteria function as vehicles to deliver the mycobacteriophage to uncapsulated (e.g., intracellular, actively replicating, infectious, e.g., in a macrophage) MAP (e.g., in an infected macrophage), where the mycobacteriophage then infect and lyse the uncapsulated MAP.

In some embodiments, a Cluster K mycobacteriophage is capable of infecting (e.g., infects) capsulated MAP and uncapsulated MAP. In some embodiments, a composition is delivered in an amount effective for lysing (e.g., kill) capsulated MAP and uncapsulated MAP. Capsulated MAP, in some embodiments, is MAP present outside of a macrophage or other immune cell. Nonencapsulate MAP, in some embodiments, is MAP present inside of a macrophage or other immune cell. Nonencapsulate MAP, in some embodiments, is active, e.g., capable of replication. Capsulated MAP, in some embodiments, is inactivated and dormant, e.g., not capable of replication. In some embodiments, uncapsulated MAP is present extracellularly, for example, in a contaminated, unpasteurized product, such as unpasteurized milk.

### Bacteriophage

A bacteriophage (phage) is a virus that infects and replicates within bacteria. In some embodiments, the compositions of the present disclosure comprise mycobacteriophage. Mycobacteriophage infect specifically mycobacteria. Non-limiting examples of a mycobacteriophage for use as provided herein are Cluster K mycobacteriophage, which infect both fast-growing and slow-growing mycobacteria (Pope W H et al. PLOS One 2011; 6(10): e26750, incorporated herein by reference). Cluster K phages have similar genomic architectures, containing a series of well conserved 13 base pair (bp) repeats associated with the translation initiation sites of a subset of the genes; approximately one half of these contain an additional sequence feature composed of imperfectly conserved 17 bp inverted repeats separated by a variable spacer. Non-limiting examples of Cluster K mycobacteriophage include mycobacteriophage TM4, mycobacteriophage Angelica, mycobacteriophage CrimD, mycobacteriophage Adephagia, mycobacteriophage Anaya, and mycobacteriophage Pixie. In some embodiments, the Cluster K mycobacteriophage is mycobacteriophage TM4.

In some embodiments, a composition comprises a mixture of two or more mycobacteriophage (e.g., Cluster K mycobacteriophage), for example, mycobacteriophage TM4 and one or more mycobacteriophage selected from mycobacteriophage Angelica, mycobacteriophage CrimD, mycobacteriophage Adephagia, mycobacteriophage Anaya, and mycobacteriophage Pixie.

### Avirulent Bacteria

Avirulent bacteria are non-pathogenic bacteria. In some embodiments, avirulent bacteria of the present disclosure are of the species Mycobacterium smegmatis. Bacteria of the species M. smegmatis are avirulent, acid-fast, gram-positive, aerobic, rod-shaped bacteria that show rapid growth and are commonly found in soil, water, and plants. A typical M. smegmatis cell has about 6.98 million nucleotides and has a high guanine-cytosine content. Naturally occurring M. smegmatis comprise about 6.7 k-6.8 k genes in a circular RNA genome that lacks plasmids. It should be understood that the composition described herein may include M. smegmatis and/or other avirulent bacteria.

In some embodiments, avirulent bacteria are transfected with mycobacteriophage. For example, M. smegmatis may be transfected with mycobacteriophage TM4. Transfection herein refers to the introduction of bacteriophage into bacterial cells. In some embodiments, mycobacteriophage infect avirulent bacteria. For example, mycobacteriophage TM4 may infect M. smegmatis. Infection, as known in the art, is a form of transfection.

### Subjects

A subject of the present disclosure, in some embodiments, is a non-human subject. For example, a subject may be a ruminant, such as a livestock animal. A ruminant is a mammal that has four compartments of its stomachs, of which the rumen is one. A livestock animal is a domesticated animal raised in an agricultural setting (e.g., on a farm) to produce labor and commodities, such as meat, eggs, milk, fur, leather, and wool. Examples of livestock animals include but are not limited to cattle, sheep, goats, camels, buffalo, pigs, horses, mules, elk, deer, and donkeys. Other livestock (e.g., farm) animals are encompassed by the present disclosure.

In some embodiments, a subject has or is at risk of Johne's disease. Johne's disease (i.e., paratuberculosis), caused by MAP, is a contagious, chronic and sometimes fatal infection that primarily affects the small intestine of ruminants. Infections normally affect ruminants but have also been seen in a variety of non-ruminant species, including pigs, rabbits, foxes, and birds. Horses, dogs, and non-human primates have been infected experimentally.

In some embodiments, a subject of the present disclosure is a human subject. MAP is capable of causing Johne's-like symptoms in humans, though it is difficult to test for MAP infection in humans, which presents a diagnostic hurdle. There are clinical similarities between Johne's disease in ruminants and inflammatory bowel disease in humans, and because of this, MAP is considered a causative agent of Crohn's disease. Thus, in some embodiments, a subject has or is at risk of Crohn's disease or other inflammatory bowel disease.

MAP has also been linked to several other disease, including juvenile sarcoidosis (Blau syndrome), Type 1 Diabetes, Autoimmune Thyroiditis, Multiple Sclerosis, Parkinson's Disease, Rheumatoid Arthritis, and Amyotrophic Lateral Sclerosis (ALS). Thus, a subject as provided herein may have or may be at risk of any one or more of the foregoing diseases.

Most ruminants, including livestock animals, are at risk of infection with MAP. A subject is considered at risk of infection with MAP if the subject consumes anything containing MAP (capsulated or uncapsulated) in food or in the environment. In some embodiments, a subject is considered at risk of infection with MAP if the subject consumes or comes into contact with ruminants, other livestock animals, or consumable commodities produced by ruminants or other livestock animals. A subject is considered to be infected with MAP if MAP can be detected in a biological sample (e.g., blood, saliva, and/or fecal sample) of the subject.

### Routes of Delivery

Compositions of the present disclosure may be delivered, for example, intravenously, intranasally, orally, subcutaneously, or intramuscularly. Other delivery routes may be used. In some embodiments, a composition is delivered to a subject intranasally, for example, as a vapor. In some embodiments, a vapor is delivered by a jet nebulizer or a soft mist inhaler (see, e.g., Carrigy N B et al. Pharm Res 2017; 34(1):2084-2096). In some embodiments, a composition is formulated as an oral tablet or capsule.

Compositions of the present disclosure, in some embodiments, are formulated as a spray. Such formulations may be applied, for example, to food or water consumed by a subject, or to ground (e.g., land, soil, and/or grass), or other surfaces contacted by a subject. In some embodiments, compositions of the present disclosure are formulated directly into water or feed/diet (e.g., dry food, liquid food, and other forms of food) of a subject.

Compositions may include one or more carriers and/or excipients. Acceptable carriers and/or excipients include, for example, natural and/or synthetic, organic and/or inorganic agents. Non-limiting examples of carriers and/or excipients include solvents, dispersion media, coatings, surfactants, antioxidants, preservatives (e.g., antibacterial agents, antifungal agents), isotonic agents, absorption delaying agents, salts, preservatives, drugs, drug stabilizers (e.g., antioxidants), gels, binders, excipients, disintegration agents, lubricants, sweetening agents, flavoring agents, and dyes, as would be known to one of ordinary skill in the art.

### Diagnostic

Also provided herein, in some embodiments, are diagnostic devices, for example a cartridge (e.g., Wang W et al. IEEE Transactions on Magnetics 2013; 49(1) and Li Y et al. J of AM Chem Soc 2010; 132:4388-4392), used to test a sample for the presence of mycobacterium, such as MAP and/or tuberculosis. In some embodiments, a mycobacterium testing device is capable of processing one or more strains and/or isolates of mycobacterium (e.g., 1, 2, 3, 4, 5, 6, 7, 8, 9, or 10 different strains and/or isolates, e.g., K-10 (cattle), MAP4 (human), MAP5 (human), MAP6 (human), 1018 (cow), 5001 (sheep), and 7560 (bison)). In some embodiments, a mycobacterium testing device is capable of testing for the presence of M. avium subsp. avium, M. avium subsp. hominissuis, M. avium subsp. Paratuberculosis, M. avium subsp. Silvaticum (see, e.g., Ghadiali A H et al. J Clin Microbiol. 2004; 42(11):5345-534; Turenne C Y et al. J Clin Microbiol 2006; 44(2):433-440; and Motiwala A S et al. Infect Immun 2006:74(11):6046-6056). A sample may be, for example, a biological sample, such as DNA obtained from blood, saliva, feces, water, or food (e.g., dairy/milk and/or meat). A mycobacterium testing device may be designed to test any combination of two or more mycobacterium strains and/or isolates provided herein.

### Additional Embodiments

Additional embodiments of the present disclosure are encompassed by the following numbered paragraphs:

1. A method comprising:

contacting a sample with a composition comprising a mycobacteriophage and/or an avirulent mycobacterium transfected with a mycobacteriophage, wherein the mycobacteriophage is capable of infecting encapsulate Mycobacterium avium subspecies paratuberculosis (MAP) and uncapsulated MAP, and wherein the sample is contaminated with MAP;

lysing extracellular and intracellular MAP in the sample.

2. The method of paragraph 1, wherein the mycobacteriophage is a Cluster K mycobacteriophage.

3. The method of paragraph 2, wherein the Cluster K mycobacteriophage is selected from the group consisting of mycobacteriophage TM4, mycobacteriophage Angelica, mycobacteriophage CrimD, mycobacteriophage Adephagia, mycobacteriophage Anaya, and mycobacteriophage Pixie.

4. The method of paragraph 3, wherein the Cluster K mycobacteriophage is mycobacteriophage TM4.

5. The method of any one of paragraphs 1-4, wherein the avirulent mycobacterium is Mycobacterium smegmatis.

6. The method of any one of paragraphs 1-5, wherein the composition comprises mycobacteriophage TM4 and Mycobacterium smegmatis transfected with mycobacteriophage TM4.

7. The method of any one of paragraphs 1-6, wherein the sample is present in water, food, ground (e.g., soil, grass, etc.) or excrement/feces.

8. The method of any one of paragraphs 1-6, wherein the sample is a tissue present in a subject.

9. The method of paragraph 8, wherein the subject is at risk of infection with MAP or is infected with MAP.

10. The method of paragraph 8 or 9, wherein the subject is a non-human subject.

11. The method of paragraph 10, wherein the non-human subject is a livestock animal.

12. The method of paragraph 11, wherein the livestock animal is selected from the group consisting of cattle, sheep, goats, camels, buffalo, pigs, horses, mules, elk, deer, and donkeys.

13. The method of any one of paragraphs 10-12, wherein the subject has or is at risk of Johne's disease.

14. The method of paragraph 8 or 9, wherein the subject is a human subject.

15. The method of paragraph 14, wherein the subject has or is at risk of Crohn's disease.

16. A method of controlling dissemination of Mycobacterium avium subspecies paratuberculosis (MAP), the method comprising applying to food and/or water a composition comprising a mycobacteriophage and/or an avirulent mycobacterium transfected with a mycobacteriophage, wherein the food and/or water is contaminated with MAP or is at risk of contamination with MAP, wherein the composition is applied in an amount effective for lysing capsulated MAP and uncapsulated MAP.

17. The method of paragraph 16, wherein the mycobacteriophage is a Cluster K mycobacteriophage.

18. The method of paragraph 17, wherein the Cluster K mycobacteriophage is selected from mycobacteriophage TM4, mycobacteriophage Angelica, mycobacteriophage CrimD, mycobacteriophage Adephagia, mycobacteriophage Anaya, and mycobacteriophage Pixie.

19. The method of paragraph 18, wherein the Cluster K mycobacteriophage is mycobacteriophage TM4.

20. The method of any one of paragraphs 16-19, wherein the avirulent mycobacterium is Mycobacterium smegmatis.

21. The method of any one of paragraphs 16-20, wherein the composition comprises mycobacteriophage TM4 and Mycobacterium smegmatis transfected with mycobacteriophage TM4.

22. The method of any one of paragraphs 16-21, wherein the food and/or water is a part of a diet of a subject.

23. The method of paragraph 22, wherein the subject is at risk of infection with MAP or is infected with MAP.

24. The method of paragraph 23, wherein the subject is a non-human subject

25. The method of paragraph 24, wherein the subject is a livestock animal.

26. The method of paragraph 24, wherein the livestock animal is selected from the group consisting of cattle, sheep, goats, camels, buffalo, pigs, horses, mules, elk, deer, and donkeys.

27. The method of any one of paragraphs 24-26, wherein the subject has or is at risk of Johne's disease

28. The method of paragraph 23, wherein the subject is a human subject.

29. The method of paragraph 28, wherein the subject has or is at risk of Crohn's disease.

30. The method of any one of paragraphs 16-29, wherein the composition is applied as a spray.

31. A method of controlling Mycobacterium avium subspecies paratuberculosis (MAP) infectivity, the method comprising:
delivering to a subject infected with MAP or at risk of infection with MAP a composition comprising a mycobacteriophage and/or an avirulent mycobacterium transfected with a mycobacteriophage, wherein the composition is delivered in an amount effective for lysing capsulated MAP and uncapsulated MAP.

32. The method of paragraph 31, wherein the mycobacteriophage is a Cluster K mycobacteriophage.

33. The method of paragraph 32, wherein the Cluster K mycobacteriophage is selected from the group consisting of mycobacteriophage TM4, mycobacteriophage Angelica, mycobacteriophage CrimD, mycobacteriophage Adephagia, mycobacteriophage Anaya, and mycobacteriophage Pixie.

34. The method of paragraph 33, wherein the Cluster K mycobacteriophage is mycobacteriophage TM4.

35. The method of any one of paragraphs 31-34, wherein the avirulent mycobacterium is Mycobacterium smegmatis.

36. The method of any one of paragraphs 31-35, wherein the composition comprises mycobacteriophage TM4 and Mycobacterium smegmatis transfected with mycobacteriophage TM4.

37. The method of any one of paragraphs 31-36, wherein the composition is delivered to the subject orally, by injection, or intranasally.

38. The method of paragraph 37, wherein the composition is delivered to the subject intranasally as a vapor, optionally wherein the vapor is delivered by a jet nebulizer or a soft mist inhaler.

39. The method of any one of paragraphs 31-38, wherein the subject is at risk of infection with MAP or is infected with MAP.

40. The method of any one of paragraphs 31-39, wherein the subject is a non-human subject.

41. The method of paragraph 40, wherein the non-human subject is a livestock animal.

42. The method of paragraph 41, wherein the livestock animal is selected from the group consisting of cattle, sheep, goats, camels, buffalo, pigs, horses, mules, elk, deer, and donkeys.

43. The method of any one of paragraphs 40-42, wherein the subject has or is at risk of Johne's disease.

44. The method of any one of paragraphs 31-39, wherein the subject is a human subject.

45. The method of paragraph 44, wherein the subject has or is at risk of Crohn's disease.

46. A composition comprising a mixture of (a) mycobacteriophage TM4 and (b) Mycobacterium smegmatis transfected with mycobacteriophage TM4, formulated for intranasal delivery in an amount effective for lysing capsulated Mycobacterium avium subspecies paratuberculosis (MAP) and uncapsulated MAP.

47. A composition comprising a mixture of (a) mycobacteriophage TM4 and (b) Mycobacterium smegmatis transfected with mycobacteriophage TM4, formulated for intravenous delivery in an amount effective for lysing capsulated Mycobacterium avium subspecies paratuberculosis (MAP) and uncapsulated MAP.

48. A composition comprising a mixture of (a) mycobacteriophage TM4 and (b) Mycobacterium smegmatis transfected with mycobacteriophage TM4, formulated as a spray in an amount effective for lysing capsulated Mycobacterium avium subspecies paratuberculosis (MAP) and uncapsulated MAP.

49. A composition comprising a mixture of (a) mycobacteriophage TM4 and (b) Mycobacterium smegmatis transfected with mycobacteriophage TM4, formulated as a feed in an amount effective for lysing capsulated Mycobacterium avium subspecies paratuberculosis (MAP) and uncapsulated MAP.

### EXAMPLES

### Example 1. Lysis of Extracellular (Uncapsulated and Capsulated) MAP Using Mycobacteriophage TM4

Active Mycobacterium avium subspecies paratuberculosis (MAP) organisms were cultured in the presence or absence of mycobacteriophage TM4 (“phage”) using standard microbiology procedures. PCR testing was performed as a secondary indicator of bacterial levels.

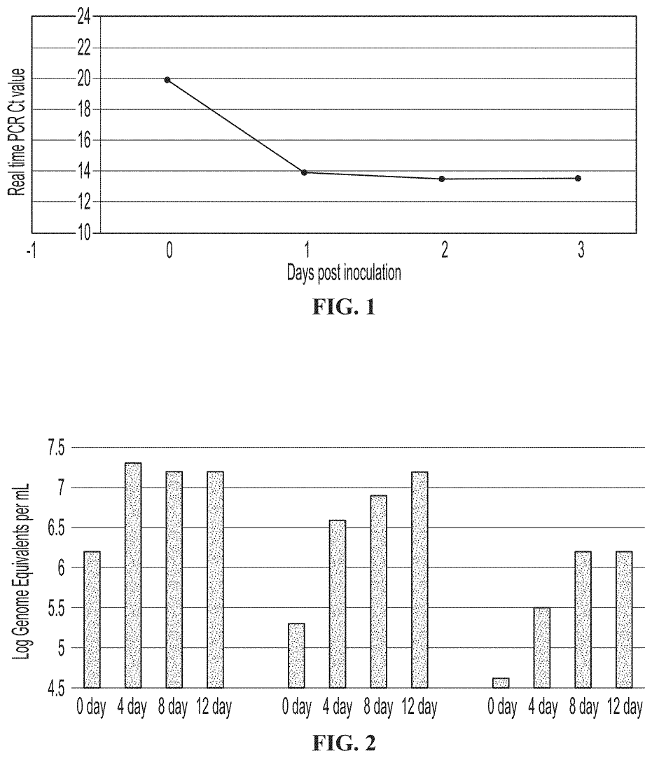

The phage used were live, could be amplified by rPCR, and were capable of infecting Mycobacterium smegmatis (M. smegmatis). FIG. 1 depicts a growth curve showing the quantification of phage by qPCR and a reduction in M. smegmatis growth. FIG. 2 depicts graphs showing active growth of three concentration of MAP-spiked control cultures in select media in TREK bottles (106, 105 and 104 bacteria/ml).

“Spiked” MAP cultures are treated with phage to measure the degree of lysing. TM4-treated MAP growth curves and AFT data are compared with control data to demonstrate the effectiveness of extracellular MAP lysis (active or dormant) by phage of various concentrations.

Active MAP cultures are laboratory stressed several days in vitro to induce capsulated MAP (inactive, dormant) for lysis by TM4.

### Example 2. Lysis of Uncapsulated, Active Intracellular MAP Using M. smegmatis Infected by Mycobacteriophage TM4

The following experiments are designed to show that M. smegmatis infected by mycobacteriophage TM4 (“phage”) is capable of introducing the phage into a macrophage, where it kills intracellular MAP. M. smegmatis infected with phage (M. smegmatis/TM4), when added to bacterium-free MAP-infected macrophage cultures, gains access to these macrophages and kills MAP in these macrophages.

Macrophages are washed and counted before infection with MAP. MAP infection is performed at a 10:1 MOI (multiplicity of infection—10 MAP to 1 macrophage). The infection is performed for 2 hours and unphagocytosed bacteria are washed out. This is T-0 for intracellular infection. At T-30 macrophage acidification is complete. At T-0 and thereafter qPCR data are taken to track active MAP growth in the macrophages and select incubation times when infection with various concentrations of M. smegmatis/TM4 will occur.

When treatments with M. smegmatis/TM4 are made, MAP carrying green fluorescent protein (GFP) is used to image and/or count the cells using fluorescence-activated cell sorting (FACS). Counting or plating of the MAP cells following macrophage lysis is used to obtain the number of surviving MAP colonies prior to PCR confirmation. This provides values of efficiency and effectiveness of MAP lysis by M. smegmatis/TM4. MAP lysis is also confirmed by real time RNA amplification (RrTPCR RNA amplification) to distinguish between dead and live cells to further substantiate results by fluorescent staining.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean±10% of the recited numerical value.

Where a range of values is provided, each value between the upper and lower ends of the range are specifically contemplated and described herein.

1. A method of controlling dissemination of Mycobacterium avium subspecies paratuberculosis (MAP), the method comprising applying to food, water, ground, and/or excrement contaminated with or at risk of contamination with MAP mycobacteriophage TM4 and Mycobacterium smegmatis cells transfected with mycobacteriophage TM4 in amounts effective for lysing capsulated MAP and uncapsulated MAP.

2. The method of claim 1, wherein the mycobacteriophage TM4 and the Mycobacterium smegmatis cells transfected with mycobacteriophage TM4 are applied to the food, water, ground, and/or excrement.

3. The method of claim 1, wherein the food and/or water are part of a diet of a subject.

4. The method of claim 1, wherein the mycobacteriophage TM4 and the Mycobacterium smegmatis cells transfected with mycobacteriophage TM4 are applied to food.

5. The method of claim 1, wherein the mycobacteriophage TM4 and the Mycobacterium smegmatis cells transfected with mycobacteriophage TM4 are applied to water.

6. The method of claim 1, wherein the mycobacteriophage TM4 and the Mycobacterium smegmatis cells transfected with mycobacteriophage TM4 are applied to ground.

7. The method of claim 7, wherein the ground comprises soil and/or grass.

8. The method of claim 1, wherein the mycobacteriophage TM4 and the Mycobacterium smegmatis cells transfected with mycobacteriophage TM4 are applied to excrement of a subject.

9. The method of claim 3, wherein the subject is a livestock animal.

10. The method of claim 9, wherein the livestock animal is selected from the group consisting of cattle, sheep, goats, camels, buffalo, pigs, horses, mules, elk, deer, and donkeys.

11. The method of claim 3, wherein the subject is at risk of infection with MAP or is infected with MAP.

12. The method of claim 3, wherein the subject has or is at risk of Johne's disease.

13. The method of claim 1, wherein the composition is applied as a spray.

14. A composition comprising a mixture of mycobacteriophage TM4 and Mycobacterium smegmatis transfected with mycobacteriophage TM4, formulated as a spray in an amount effective for lysing capsulated Mycobacterium avium subspecies paratuberculosis (MAP) and uncapsulated MAP.

15. A composition comprising a mixture of mycobacteriophage TM4 and Mycobacterium smegmatis transfected with mycobacteriophage TM4, formulated as a feed in an amount effective for lysing capsulated Mycobacterium avium subspecies paratuberculosis (MAP) and uncapsulated MAP.
